(19) United States
(12) Patent Application Publication
(10) Pub. No.: US 2020/0216788 A1
(43) Pub. Date: Jul. 9, 2020

(54) KOJI PRODUCTION SYSTEM

(71) Applicant: YAMASA CORPORATION, Choshi-shi (JP)

(72) Inventors: Yoshiyuki TOYOSHIMA, Choshi-shi (JP); Yasunori MATSUURA, Choshi-shi (JP)

(73) Assignee: YAMASA CORPORATION, Choshi-shi (JP)

(21) Appl. No.: 16/737,195

(22) Filed: Jan. 8, 2020

(30) Foreign Application Priority Data
Jan. 9, 2019 (JP) 2019-001920

(51) Int. Cl. C12M 1/16; C12M 1/34; C12M 1/36

(52) U.S. Cl. CPC C12M 21/16; C12M 41/48; C12M 41/12; C12M 41/32

(57) ABSTRACT

A Koji production system including a culture area, an environment control device, a Koji container, a carrying-in lane, a Koji container moving device, and a control unit. The culture area is an area for culturing the Koji by storing a plurality of the Koji container in a predetermined position. The environment control device is configured to control a temperature and humidity in the culture area. The Koji container contains raw materials of Koji placed at a height of 1 cm to 10 cm, the raw materials comprising a mixture of soybeans and Koji mold. The carrying-in lane is configured to move the Koji container to the culture area.

### TECHNICAL FIELD

The present invention relates to a Koji Production system.

### BACKGROUND ART

Techniques aimed at improving the efficiency of the production of Koji used for the production of soy sauce have been proposed.

Patent Literature 1 discloses a Koji making room device where a fan, a cooler and a heater are provided in a room and the temperature in the room can be adjusted.

### CITATION LIST

### Patent Literature

Patent Literature 1: Japanese Patent Publication No. S30-9144

### SUMMARY OF INVENTION

### Technical Problem

The present invention provides a Koji making system capable of realizing further efficiency in the production of Koji.

### Solution to Problem

According to the present invention, provided is a Koji production system comprising a culture area, an environment control device, a Koji container, a carrying-in lane, a Koji container moving device, and a control unit. The culture area is an area for culturing the Koji by storing a plurality of the Koji container in a predetermined position. The environment control device is configured to control a temperature and humidity in the culture area. The Koji container contains raw materials of Koji placed at a height of 1 cm to 10 cm, the raw materials comprising a mixture of soybeans and Koji mold. The carrying-in lane is configured to move the Koji container to the culture area. The Koji container moving device is configured to move the Koji container on the carrying-in lane to the predetermined position, the Koji container being installed into the culture area. The control unit controls the environment control device so as to vary the temperature and the humidity in the culture area according to the type of the Koji mold or the raw materials.

According to the present invention, when storing a Koji container containing raw materials of Koji comprising a mixture of soybeans and Koji molds at a height of 1 cm to 10 cm in a culture area, the controller varies the temperature and the humidity in the culture area according to the type or raw materials of Koji molds. As a result, the raw materials can be stored at an optimum temperature and humidity according to the type or raw materials of the Koji mold, and since the thickness of the raw materials is 1 cm to 10 cm, the temperature of the raw materials can be kept substantially uniform and it becomes possible to efficiently produce good Koji.

Hereinafter, various embodiments of the present invention will be exemplified. The following embodiments can be combined with each other.

The culture area preferably includes an external air flow inlet through which external air can flow in, and the control unit controls the external air flow inlet to vary the oxygen concentration in the culture area by adjusting the inflow amount of the external air flowing into the culture area according to the temperature, humidity or oxygen concentration in the culture area or the temperature of the Koji stored in the culture area.

The control unit preferably controls the environment control device to switch the temperature and the humidity fluctuation ratio in the culture area according to a type of the Koji mold or the raw materials.

The control unit preferably controls the environment control device such that the temperature in the culture area is changed from a range of 25° C. to 35° C. into a range of 15° C. to 24° C., and the humidity in the culture area is changed from a range of 70% to 100% into a range of 30% to 70% within 72 hours from a start of storage of the raw materials.

When the raw materials are stored in the culture area, the control unit preferably determines a timing for carrying raw materials which are different from the raw materials into the culture area, according to the type of Koji mold.

An air blower capable of blowing a substantially parallel wind in the culture area is preferably provided, wherein the control unit controls the air blower such that the temperature of the raw materials in the culture area may becomes substantially uniform.

A storage shelf is preferably provided in the culture area, a plurality of the raw materials is stored in the storage shelf, and the storage shelf is configured such that the air blown by the air blower can pass through the storage shelf.

A discharge device for discharging the raw material is preferably provided, wherein the discharge device discharges the raw materials into the Koji container so that the height of the raw material becomes 1 cm to 10 cm.

A plurality of the Koji containers is preferably stored in the culture area in the vertical direction.

### DESCRIPTION OF THE DRAWINGS

FIG. 1 is a schematic view of a Koji production system 100 according to an embodiment of the present invention. Here, in FIG. 1, the culture area 1 is shown as a front view, and a carrying-in lane 4 and a carrying-out lane 5 are shown as a plan view.

FIG. 2 is a schematic diagram illustrating an air inlet hole 32 provided in a bottom 31 of the Koji container 3.

FIG. 3 is an example of a table that prescribes control of temperature and humidity in the culture area 1 defined by each type of Koji mold.

FIG. 4 is a graph diagram showing the timing on carrying a different type of raw material into the culture area 1 according to the type of Koji mold.

### DESCRIPTION OF EMBODIMENTS

Hereinafter, embodiments of the present invention will be described with reference to the drawings. The characteristic matters shown in the following embodiments can be combined with each other.

### 1. First Embodiment

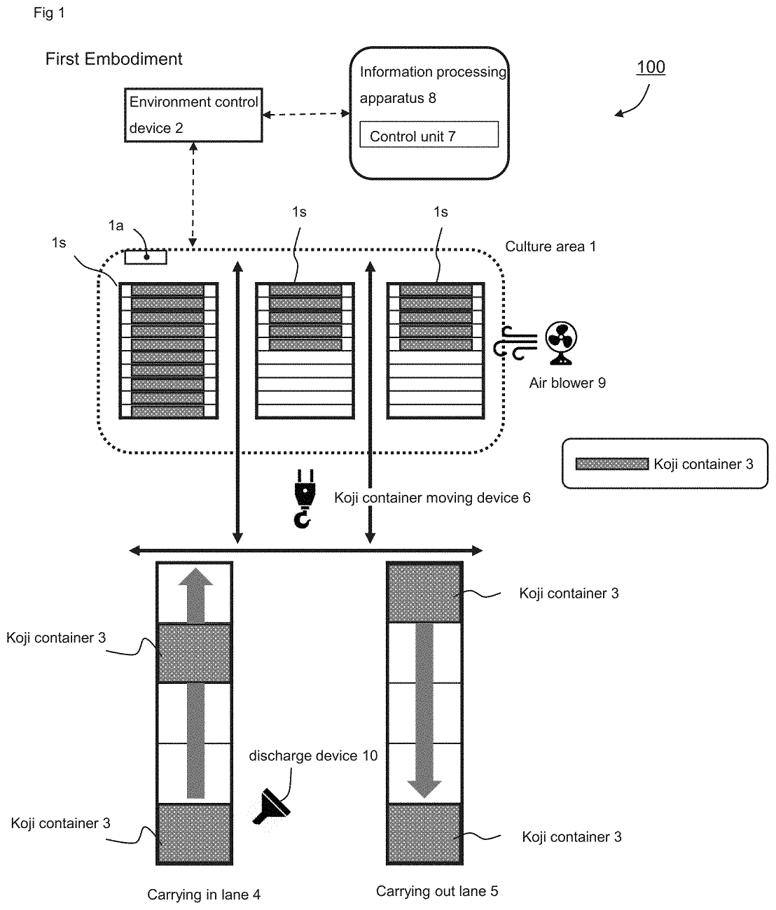

Hereinafter, a Koji production system 100 according to the first embodiment of the present invention will be described with reference to FIG. 1 to FIG. 3. The Koji production system 100 can efficiently produces Koji used for the making of brewed foods such as soy sauce and miso. Specifically, such Koji is produced by growing Koji mold in Koji raw materials comprising at least a mixture of soybean and Koji mold.

As shown in FIG. 1, the Koji production system 100 includes a culture area 1, an environment control device 2, a Koji container 3, a carrying-in lane 4, a Koji container moving device 6, and a control unit 7. Furthermore, in this embodiment, the carrying-out lane 5, the storage shelf 1 s, the air blower 9, and the information processing apparatus 8 are provided.

The culture area 1 is an area where a plurality of Koji container 3 are stored at predetermined positions to culture Koji. In the present embodiment, the culture area 1 is a space whose periphery is covered, and includes an external air flow inlet 1a through which external air can flow in. The amount of external air flowing into the culture area 1 can be adjusted by opening or closing the external air flow inlet 1a. In the present embodiment, the oxygen concentration in the culture area 1 can be adjusted by adjusting the amount of external air flowing into the culture area 1. Further, a storage shelf 1s is provided in the culture area 1. And the plurality of raw materials is stored on the storage shelf 1s. In the present embodiment, three storage shelves 1s that can store ten Koji containers 3 disposed vertically are provided. Therefore, a plurality of Koji containers 3 are stored in the culture area 1 in the vertical direction. Note that the number of stages, size, number, position, and the like of the storage shelf 1s are not particularly limited, and may be determined as appropriate.

The environment control device 2 is configured to be capable of controlling the temperature and the humidity in the culture area 1. Specifically, the environment control device 2 includes a temperature adjustment device and a humidity adjustment device. Furthermore, in this embodiment, the air blower 9 is provided which can ventilate the substantially parallel wind in the culture area 1. The number of the air blowers 9 is not particularly limited, and may be one as shown in FIG. 1, may be two provided at opposite locations of the culture area 1, or may be three or more. Here, the storage shelf 1s is configured such that the air blown by the air blower 9 can pass through the storage shelf 1s. For example, an opening can be provided on the side surface of the storage shelf 1s, or the storage shelf 1s may have rack shape. Thereby, the air from the air blower 9 also reaches the storage shelf 1s located in the center of the culture area 1.

The Koji container 3 is a container in which raw materials of Koji comprising a mixture of soybeans and Koji molds is placed vertically at a height of 1 cm to 10 cm. Preferably, the height of the raw materials is 1.5 cm to 8 cm, more preferably 1.7 cm to 6 cm, and more preferably 1.9 cm to 4 cm. In terms of well growth of Koji mold, it is desirable that the height of the raw material is low, but it is not preferable in terms of production efficiency. On the other hand, in terms of production efficiency, the height of the raw material is preferably as high as possible. However, if the height of the raw materials exceeds 10 cm, heat accumulates in the center of the raw materials placed in the Koji container 3 and there is a possibility that the temperature may be exceeded the temperature suitable for Koji production. Thus, in this embodiment, the raw materials of the Koji are placed in the Koji container 3 vertically at a height of 1 cm to 10 cm.

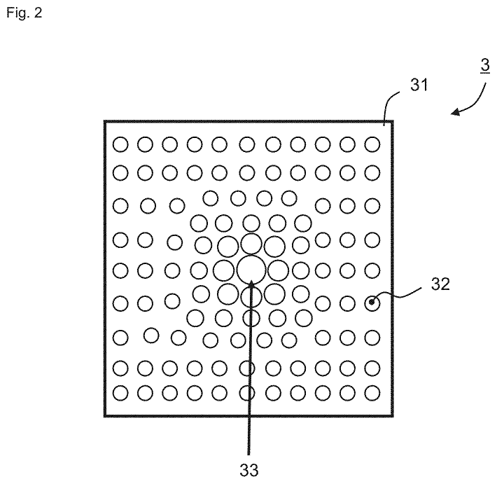

As shown in FIG. 2, the Koji container 3 includes a bottom 31 on which raw materials can be placed. The bottom 31 is provided with a plurality of air inlet holes 32. The air inlet holes 32 may be provided such that the density in the central portion 33 of the bottom 31 is maximized. Further, the size of the air inlet hole 32 can be configured to be maximized in the central portion 33 of the bottom 31. The raw materials of Koji have low thermal conductivity, and heat tends to accumulate in the raw material placed near the central portion 33 of the Koji container 3. Thus, it is preferable that air can be easily passed through the central portion 33. In this way, the temperature of the raw materials placed in the central portion 33 and other regions is made substantially uniform by lowering the temperature of the central portion 33.

The Koji container moving device 6 is configured to move the Koji container 3 on the carrying-in lane 4 to a predetermined position in the culture area 1 and the Koji container 3 can be carried into the culture area 1. In the present embodiment, the Koji container moving device 6 is configured to be movable in the direction in which the carrying-in lane 4 and the carrying-out lane 5 are arranged in the culture area 1 and to be movable in the vertical direction along the storage shelf 1s. Specifically, the Koji container 3 on the carrying-in lane 4 are taken out one by one, and the Koji container 3 are installed on a predetermined position of the storage shelf 1s provided in the culture area 1. Here, the configuration of the Koji container moving device 6 is not particularly limited, and is realized by, for example, a crane or a mobile lifting device.

The control unit 7 controls the environment control device 2 so as to vary the temperature and the humidity in the culture area 1 according to the type of the Koji mold or raw materials. In the present embodiment, the control unit 7 is stored in an information processing apparatus 8 such as a computer. Further, the information processing apparatus 8 includes a transmission unit (not shown), inputs the type of raw materials which will be stored in the culture area 1 to the control unit 7 via the transmission unit, and outputs the control signal to the environment control device 2.

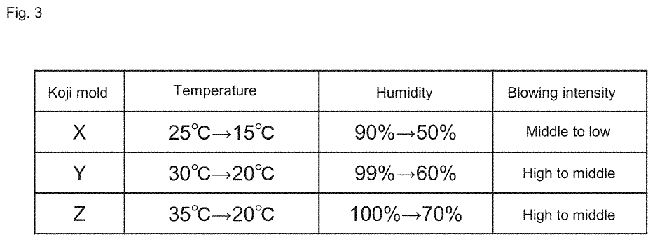

As shown in FIG. 3, the control unit 7 controls the environment control device 2 so as to switch the fluctuation ratio of the temperature and the humidity in the culture area 1 according to the type of the Koji mold. In this embodiment, the control unit 7 controls the environment control device 2 such that the temperature in the culture area 1 is changed from a range of 25° C. to 35° C. into a range of 15° C. to 24° C. and the humidity in the culture area 1 is changed from a range of 70% to 100% into a range of 30% to 70% within 72 hours from a start of storage of the raw materials. The temperature is preferably controlled from a range of 27° C. to 33° C. into a range of 17° C. to 22° C., more preferably from a range of 29° C. to 31° C. into a range of 19° C. to 20° C. Further, the humidity is preferably controlled from a range of 75% to 99.9% into a range of 35% to 65%, and more preferably from a range of 80% to 99% into a range of 40% to 60%. For example, when the raw materials containing Koji mold X is stored in the culture area 1, the environment control device 2 is controlled such that the temperature in the culture area 1 is 25° C. and the humidity is 90% before the Koji container 3 is carried into the culture area 1, and the temperature in the culture area 1 becomes 15° C. and the humidity becomes 50% after 72 hours have passed from the carrying the Koji container 3 into the culture area 1. Such fluctuation ratios of the temperature and the humidity are appropriately determined according to the type of Koji mold, and such ratios may be, for example, a linear decrease or a discontinuous non-linear decrease.

Moreover, in this embodiment, the control unit 7 controls the air blower 9 such that the temperature of the raw materials in the culture area 1 may become substantially uniform. In the example of FIG. 3, when the raw materials containing Koji mold X is stored in the culture area 1, the air blowing intensity of the air blower 9 is set to “medium” before the Koji container 3 is carried into the culture area 1, and the air blowing intensity of the air blower 9 is set to “low” after 48 hours have passed from the carrying the Koji container 3 into the culture area 1. In addition to the environment control device 2, it is easy to make the temperature of the raw material placed in the Koji container 3 substantially uniform by sending the air with the air blower 9.

Similarly, when the raw materials containing Koji mold Y is stored in the culture area 1, the environment control device 2 is controlled such that the temperature in the culture area 1 is 30° C. and the humidity is 99% before the Koji container 3 is carried into the culture area 1, and the temperature in the culture area 1 becomes 20° C. and the humidity becomes 60% after 48 hours have passed from the carrying the Koji container 3 into the culture area 1. Also, when the raw materials containing Koji mold Z is stored in the culture area 1, the environment control device 2 is controlled such that the temperature in the culture area 1 is 35° C. and the humidity is 100% before the Koji container 3 is carried into the culture area 1, and the temperature in the culture area 1 becomes 20° C. and the humidity becomes 70% after 48 hours have passed from the carrying the Koji container 3 into the culture area 1.

Here, the temperature and humidity described above are examples, and may be determined suitably according to the type of Koji mold and height of the raw materials mounted in the Koji container 3. Further, the temperature and the humidity in the culture area 1 can be controlled to change to predetermined values after not only 72 hours but also a predetermined time has passed since the Koji container 3 was carried into the culture area 1.

In the example of FIG. 3 described above, the control unit 7 controls the environment control device 2 according to the type of Koji mold, the environment control device 2 can be controlled to switch the fluctuation ratio of the temperature and the humidity in the culture area 1 according to the raw materials of Koji. In this case, a table similar to FIG. 3 may be prepared for each blend of raw materials.

Further, in the present embodiment, the control unit 7 controls the external air flow inlet 1a to vary the oxygen concentration in the culture area 1 by adjusting the inflow amount of the external air flowing into the culture area 1 according to the temperature, humidity or oxygen concentration in the culture area 1 or the temperature of the Koji stored in the culture area 1. This is because oxygen is necessary for the growth of Koji mold. For example, when the growth of Koji mold is too vigorous and the temperature of the Koji rises too much, the inflow of external air is reduced (or the inflow of external air is stopped) to suppress excessive growth of Koji. Here, the temperature of Koji is measured by an arbitrary temperature sensor, the temperature is transmitted to the control unit 7, and the control unit 7 controls the external air flow inlet 1a. Also, the same adjustment as described above may be performed when the temperature or the humidity in the culture area 1 is high or when the oxygen concentration in the culture area 1 is high. Here, the predetermined temperature can be appropriately determined depending on the type or raw materials or Koji mold. The external air flow inlet 1a may be connected to the air blower 9 as necessary.

Then, after Koji mold sufficiently grows in the culture area 1 and becomes Koji that can be used for producing brewed foods such as soy sauce or miso, the Koji container 3 is taken out from the storage shelf is by the Koji container moving device 6 and is carried out to the carrying-out lane 5. Thereafter, for example, when producing soy sauce, Koji placed in the Koji container 3 is taken out, salt dissolved water is added and stored in a storage tank. And soy sauce is completed through the processes such as fermentation, maturing, filter pressing, pasteurizing and inspection.

As described above, in the Koji production system 100 according to the first embodiment, the raw materials are discharged into the Koji container 3 by the discharge device 10 so that the height of the raw materials becomes 1 cm to 10 cm. Then, the Koji container 3 is moved toward the culture area 1 by the carrying-in lane 4, and the Koji container moving device 6 transports the Koji container 3 to a predetermined position of the storage shelf is of the culture area 1. After the plurality of Koji containers 3 are carried into the storage shelf 1s, the temperature and the humidity in the culture area 1 are controlled by the control unit 7 according to the type of Koji mold or raw materials of Koji. At this time, it becomes possible to control the temperature and the humidity of the culture area 1 by the environment control device 2 and to control the temperature and the humidity with higher accuracy by the air blower 9. Furthermore, when the growth of the Koji is excessive, the control unit 7 can suppress the excessive growth of Koji by adjusting the oxygen concentration in the culture area 1. Thereby, the first embodiment has advantageous effect that the manual process is unnecessary for stirring raw materials to make the temperature of raw materials uniform. Moreover, since this control unit 7 controls the temperature and the humidity in the culture area 1 according to the type of Koji mold or raw materials of Koji, it becomes easy to produce the optimum Koji according to various products.

Furthermore, in the first embodiment, since the height of the raw materials is set to 1 cm to 10 cm, the height of the Koji container 3 can be lowered, so that it becomes easy to store a plurality of Koji containers 3 arranged in the vertical direction in the culture area 1. Thereby, a large amount of raw materials can be stored in the culture area 1.

Furthermore, by attaching an identification number to the container 3 and linking it to the final product, it becomes easy to trace the production lot of Koji. Here, the attaching of the identification number to the Koji container 3 is realized by attaching an arbitrary two-dimensional code including information indicating the identification number to the Koji container 3. Also, by changing the type of Koji mold and the composition of raw materials for each Koji container 3, it becomes possible to produce a large variety of Koji even by a single production.

### 2. Second Embodiment

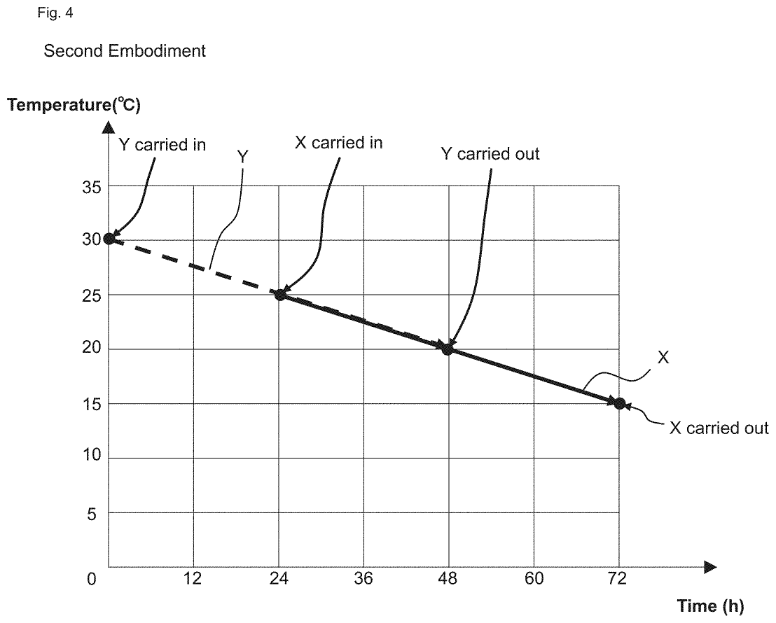

Next, the Koji production system 100 according to the second embodiment of the present invention will be described with reference to FIG. 4. Since the structure of the Koji production system 100 in second embodiment is the same as that of the first embodiment, the description is omitted.

In the second embodiment, when the raw materials are stored in the culture area 1, the control unit 7 determines a timing for carrying a different type of raw materials (a raw material containing different types of Koji molds) in the culture area 1 according to the type of Koji mold.

For example, as shown in FIG. 4, when the temperature in the culture area 1 is linearly decreased with respect to the raw materials containing the Koji mold X and Y, the Koji mold Y (denoted as Y in the figure) is carried in the culture area 1. Here, the storage shelf is leaves a space for storing the Koji container 3. At this time, the temperature in the culture area 1 is set to 30° C. by the control unit 7. After this, if the raw material containing Koji mold X (indicated as X in the figure) is stored in the culture area 1 to produce Koji due to a change in the production plan etc., the production manager inputs an instruction into the information processing apparatus 8 to carry the raw materials containing Koji mold X to the culture area 1.

Here, as shown in FIG. 3, the raw materials containing Koji mold X is to be carried in the culture area 1 when the temperature of the culture area 1 is 25° C. In order to realize this, the control unit 7 receives the time after all the raw materials containing Koji mold Y are carried into the culture area 1, and specifies the current temperature based on the table of FIG. 3. In the present embodiment, the temperature becomes 25° C. after 24 hours has passed since all the raw materials containing Koji mold Y are carried into culture area 1, the raw materials containing Koji mold X is carried into culture area 1 at this time.

This makes it possible to simultaneously store raw materials containing different types of Koji molds in one culture area 1.

In addition, as shown in FIG. 3, the raw materials containing Koji mold Z is to be carried into the culture area 1 under the condition in which the temperature of the culture area 1 is 35° C. For this reason, a warning may be issued by the control unit 7, when the raw materials containing Koji mold Y is stored with the temperature in the culture area 1 lowered from 30° C. and the raw materials containing Koji mold Z is carried into the culture area 1.
1: culture area
1s: storage shelf
1a: external air flow inlet
2: environment control device
3: Koji container
31: bottom
32: air inlet hole
33: central part
4: carrying-in lane
5: carrying-out lane
6: Koji container moving device
7: control unit
8: information processing device
9: air blower
10: discharge device
100: Koji production system

1. A Koji production system comprising:
a culture area, an environment control device, a Koji container, a carrying-in lane, a Koji container moving device, and a control unit,
wherein the culture is an area for culturing the Koji by storing a plurality of the Koji container in a predetermined position,
the environment control device is configured to control a temperature and a humidity in the culture area,
the Koji container contains raw materials of Koji placed at a height of 1 cm to 10 cm, the raw materials comprising a mixture of soybeans and Koji mold,
the carrying-in lane is configured to move the Koji container to the culture area,
the Koji container moving device is configured to move the Koji container on the carrying-in lane to the predetermined position, the Koji container being installed into the culture area, and
the control unit controls the environment control device so as to vary the temperature and the humidity in the culture area according to the type of the Koji mold or the raw materials.

2. The system of claim 1, wherein the culture area includes an external air flow inlet through which external air can flow in, and
the control unit controls the external air flow inlet to vary the oxygen concentration in the culture area by adjusting the inflow amount of the external air flowing into the culture area according to the temperature, humidity or oxygen concentration in the culture area or the temperature of the Koji stored in the culture area.

3. The system of claim 1, wherein the control unit controls the environment control device to switch the temperature and the humidity fluctuation ratio in the culture area according to a type of the Koji mold.

4. The system of claim 1, wherein the control unit controls the environment control device such that the temperature in the culture area is changed from a range of 25° C. to 35° C. into a range of 15° C. to 24° C., and the humidity in the culture area is changed from a range of 70% to 100% into a range of 30% to 70% within 72 hours from a start of storage of the raw materials.

5. The system of claim 1, wherein when the raw materials are stored in the culture area, the control unit determines a timing for carrying raw materials which are different from the raw materials in the culture area according to the type of Koji mold.

6. The system of claim 1, further comprising:
an air blower capable of blowing a substantially parallel wind in the culture area,
wherein the control unit controls the air blower such that the temperature of the raw materials in the culture area may become substantially uniform.

7. The system of claim 6, wherein a storage shelf is provided in the culture area,
a plurality of the raw materials is stored in the storage shelf, and
the storage shelf is configured such that the air blown by the air blower can pass through the storage shelf

8. The system of claim 1, further comprising:
a discharge device for discharging the raw material, wherein
the discharge device discharges the raw materials into the Koji container so that the height of the raw material becomes 1 cm to 10 cm.

9. The system of claim 1, wherein a plurality of the Koji containers is stored in the culture area in the vertical direction.
